(19) United States
(12) Patent Application Publication
(10) Pub. No.: US 2023/0012300 A1
(43) Pub. Date: Jan. 12, 2023

(54) LIGHT INTEGRATED DEVICES WITH DUAL LIGHT EMITTING DIODES

(71) Applicant: BioLight, Inc., Stevensville, MT (US)

(72) Inventor: Michael BELKOWSKI, Stevensville, MT (US)

(73) Assignee: BioLight, Inc., Stevensville, MT (US)

(21) Appl. No.: 17/810,681

(22) Filed: Jul. 5, 2022

Related U.S. Application Data
(60) Provisional application No. 63/218,709, filed on Jul. 6, 2021.

(51) Int. Cl. H05B 47/165; H05B 45/10; H05B 45/20; H05B 47/17; H05B 47/105; H05B 47/155; F21V 23/00; F21V 23/04; G06N 20/00

(52) U.S. Cl. CPC H05B 47/165; H05B 45/10; H05B 45/20; H05B 47/17; H05B 47/105; H05B 47/155; F21V 23/003; F21V 23/0442; G06N 20/00; F21Y 2115/10

(57) ABSTRACT

Systems and methods are disclosed for configuring a light integrated device, and include receiving sensed data from a first sensor, providing the sensed data from the first sensor to a machine learning model, receiving a machine learning output from the machine learning model based on the sensed data from the first sensor, the machine learning output comprising a light integrated device configuration, wherein the light integrated device configuration comprises a dual light emitting diodes (LED) setting, and configuring the light integrated device based on the light integrated device configuration.

### CROSS-REFERENCE TO RELATED APPLICATIONS

This application claims the benefit of U.S. Provisional Patent Application 63/218,709, filed Jul. 6, 2021, the entire content of which is incorporated herein by reference.

### TECHNICAL FIELD

Various embodiments of the present disclosure relate generally to integrated light devices and, more particularly, to devices configured to provide dual light emitting diode (LED) based red and near-infrared light therapy to a user.

### BACKGROUND

Light therapy such as sunlight exposure, vitamin D therapy, and the like have been traditionally used for their health benefits. However, such exposure can often be uncontrolled and may lead to conditions such as over-exposure, sunburns, and can result in increased risk of developing chronic conditions as a result of the exposure. In-home solutions provide a limited range of therapies, often with unsafe emission values and limited therapeutic benefits.

### SUMMARY OF THE DISCLOSURE

According to certain aspects of the disclosure, methods and systems are disclosed for using light integrated devices.

According to an aspect disclosed herein, a method for configuring a light integrated device includes receiving sensed data from a first sensor; providing the sensed data from the first sensor to a machine learning model; receiving a machine learning output from the machine learning model based on the sensed data from the first sensor, the machine learning output comprising a light integrated device configuration, wherein the light integrated device configuration comprises a dual light emitting diodes (LED) setting; and configuring the light integrated device based on the light integrated device configuration.

According to another aspect disclosed herein, a light integrated device includes a housing; one or more sensors associated with the housing; one or more dual light emitting diodes (LEDs) associated with the housing, wherein each dual LED is configured to output a first light having a first wavelength and a second light having a second wavelength; and a processor configured to cause the one or more dual LEDs to operate based on a light integrated device configuration.

According to another aspect disclosed herein, a system for providing light therapy to a user includes one or more sensors configured to sense sensed data; a light integrated device comprising one or more dual light emitting diodes (LEDs), wherein each dual LED is configured to output red light and near red light; at least one memory storing instructions; and at least one processor executing the instructions to perform a process, the processor configured to: receive the sensed data sensed by the one or more sensors; receive a light integrated device configuration based on the sensed data, the light integrated device configuration comprising one or more of wavelengths of light, intensities of light, rates, durations, or frequencies for configuring the light integrated device; and configure the light integrated device based on the light integrated device configuration.

It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1A depicts a table top light panel, according to one or more embodiments.

FIG. 1B depicts a cylindrical light panel, according to one or more embodiments.

FIG. 2 depicts a table top light panel with different emissions, according to one or more embodiments.

FIGS. 3A-3I depict a plurality of table top light panel arrangements, according to one or more embodiments.

FIG. 4 depicts a full body light panel, according to one or more embodiments.

FIG. 5A depicts a diagram of light penetration from a red light emitted from a light panel, according to one or more embodiments.

FIG. 5B depicts a diagram of light penetration from a near infrared light emitted from a light panel, according to one or more embodiments.

FIGS. 6A, 6B, 6C, 6D, and 6E depict sensors and dual light emitting diodes associated with integrated light devices, according to one or more embodiments.

FIG. 7A depicts a flow chart for targeted light therapy, according to one or more embodiments.

FIG. 7B depicts a system environment for targeted light therapy, according to one or more embodiments.

FIG. 8 depicts a data flow for training a machine learning model, according to one or more embodiments.

FIG. 9 depicts an example system that may execute techniques presented herein.

### DETAILED DESCRIPTION OF EMBODIMENTS

The terminology used herein may be interpreted in its broadest reasonable manner, even though it is being used in conjunction with a detailed description of certain specific examples of the present disclosure. Indeed, certain terms may even be emphasized herein; however, any terminology intended to be interpreted in any restricted manner will be overtly and specifically defined as such in this Detailed Description section. Both the general description and the detailed description are exemplary and explanatory only and are not restrictive of the features, as claimed.

As used herein, the terms “comprises,” “comprising,” “having,” including,” or other variations thereof, are intended to cover a non-exclusive inclusion such that a process, method, article, or apparatus that comprises a list of elements does not include only those elements, but may include other elements not expressly listed or inherent to such a process, method, article, or apparatus.

In this disclosure, relative terms, such as, for example, “about,” “substantially,” “generally,” and “approximately” are used to indicate a possible variation of ±10% in a stated value.

The term “exemplary” is used in the sense of “example” rather than “ideal.” As used herein, the singular forms “a,” “an,” and “the” include plural reference unless the context dictates otherwise.

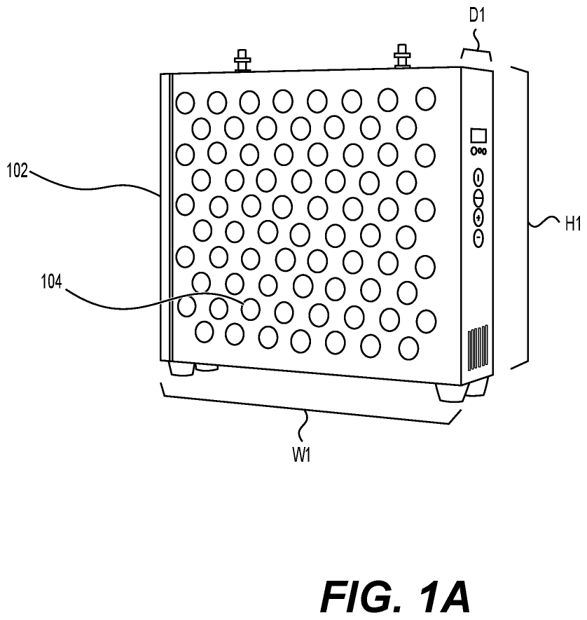

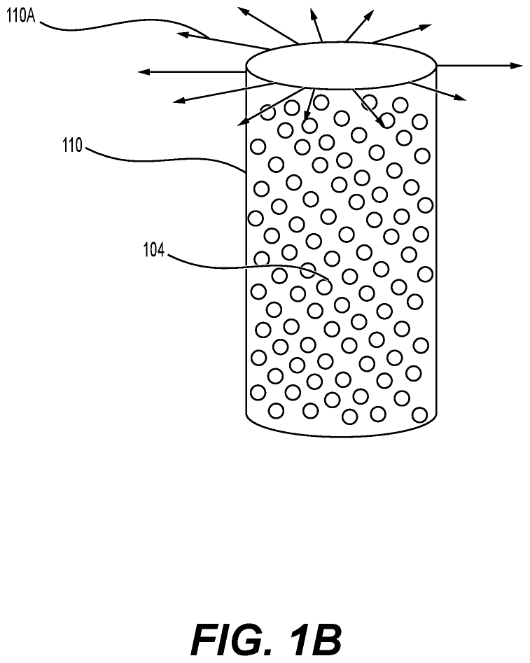

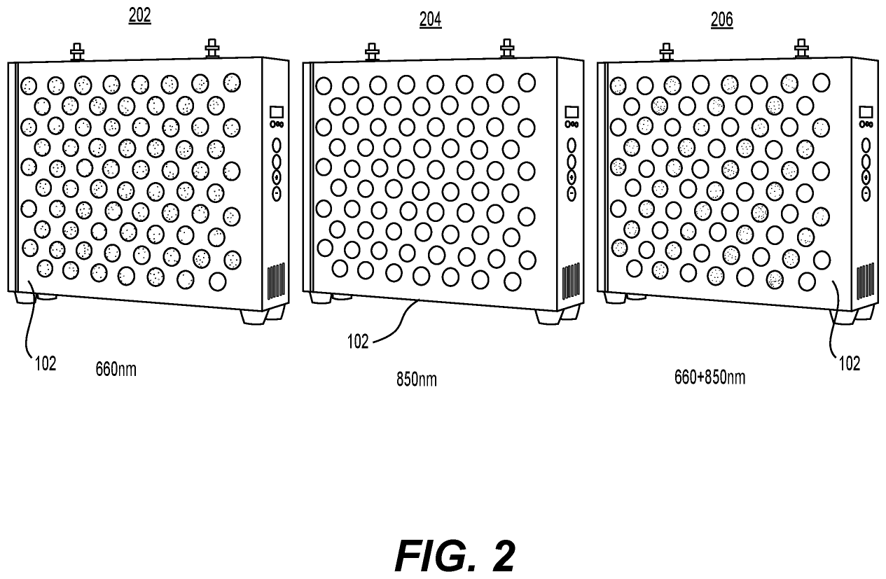

According to implementations of the disclosed subject matter, a light panel 102 is provided herein, as shown in FIG. 1A. Light panel 102 may include a plurality of dual LEDs 104. Light panel 102 may be configured to selectively provide red light (e.g., with a wavelength of approximately 630 nm, or within a range of approximately 600 nm-750 nm), near infrared light (e.g., with a wavelength of approximately 850 nm, or within a range of approximately 750 nm-1000 nm), or a combination thereof. Generally, light panel 102 may be configured to provide light in the range of approximately 600 nm-1000 nm. For example, FIG. 2 shows light panel 102 emitting red light at approximately 660 nm at 202, near infrared light at approximately 850 nm at 204, and a combination of red light at approximately 660 nm and near infrared light at approximately 850 nm at 206. It will be understood that when emitting either red light or near infrared light, each of the dual LEDs 104 may emit the respective frequency of lights (e.g., not a subset of the dual LEDs 104).

Light panel 102 may be used to promote health and/or treat or mitigate health conditions such as inflammatory conditions, hair health, pain, mental health, sleep conditions, thyroid health, athletic performance, or the like. Light panel 102, based on the use of both the red light and the near infrared light, may reduce inflammation, increase circulation, and optimize the functionality of mitochondria (e.g., to allow cells to generate energy more efficiently). As further discussed herein, the combination of the red light and near infrared light may be used to treat both surface conditions and sub-surface conditions (e.g., at a tissue level).

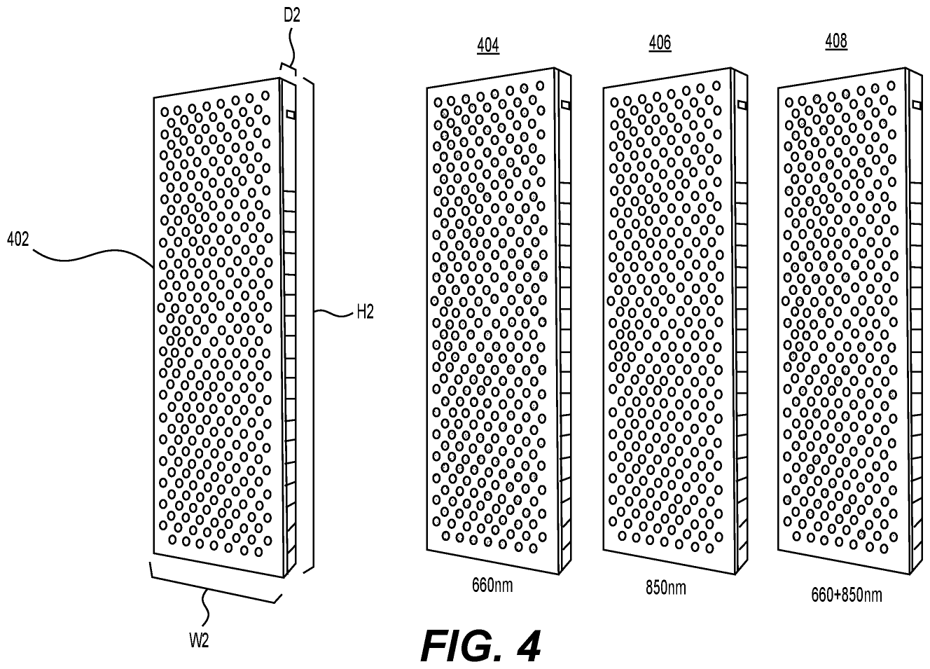

Light panel 102 may be configured to emit light towards a subject (e.g., a user). Light panel 102 may be a flat panel, such as the table top light panels shown in FIGS. 1A, 2, and 3A-3I, or a full body light panel 402 shown in FIG. 4. Alternatively, the one or more of the light panels disclosed herein may be curved or otherwise arranged to direct light emission towards a subject. For example, a curved light panel may direct light towards a center point of the curve created by the curved light panel.

FIG. 1A shows a height H1 for light panel 102. The height H1 may be, for example, approximately 11.8 inches. FIG. 1A also shows a width W1 for light panel 102. The width W1 may be, for example, approximately 11 inches. FIG. 1A shows a depth D1 for light panel 102. The depth D1 may be, for example, 2.6 inches. It will be understood that light panel 102 may be of variable heights, widths, and depths, other than those disclosed herein.

According to an implementation, light panel 102 may emit light from two or more sides. For example, a light panel may include dual LEDs 104 on two or more sides of the light panel. A light panel that includes dual LEDs 104 on two sides may be used by two users, one user on each side of the light panel. A light pane that includes LEDs on three or more sides, or, as shown in FIG. 1B, a cylindrical light panel 110 may be placed between multiple users such that each user receives a light emission. As shown, the cylindrical light panel 110 may include dual LEDs 104 that emit light in multiple directions (e.g., 360 degrees, as indicated by arrows 110A) such that users around the cylindrical light panel 110, at any angle, may receive its emission. As an example, a table top light panel (e.g., cylindrical light panel 110) that emits light from multiple sides may be placed on a table for users to receive light therapy while seated around the table.

It will be understood that the subject matter disclosed herein in reference to light panel 102 generally applies to curved or light panels that emit light in multiple directions (e.g., cylindrical light panel 110). Light panel 102 may be formed from any applicable material that maintains the structure of light panel 102. The material may dissipate heat to a degree such that a user that comes in contact with light panel 102 during its use or after its use is not harmed by the temperature of light panel 102.

Light panel 102 may include one or more input receptors which may be knobs, buttons, touch points (e.g., haptic response points), or the like. Alternatively or in addition, light panel 102 may be voice or gesture activated. The input receptors may include a power button to power light panel 102 on and off, may include a timer, may include a setting adjustor, or the like. According to an implementation, two or more tasks may be performed by the same input receptor (e.g., power and timer operation may be conducted using the same button). The input receptors may be used to adjust a configuration of light panel 102 which may include a wavelength, an intensity, a duration, or the like. The configurations may be output by a machine learning model, as further discussed herein, and may be based on one or more sensed health conditions (e.g., a hair treatment setting, a muscle treatment setting, etc.). The configurations may configure light panel 102 by adjusting one or more of a wavelength, intensity, duration, or the like and/or may further be based on selection of a treatment modality.

Light panel 102 may include a timer configured to automatically shut-off light panel 102 after a given amount of time. The given amount of time may be pre-determined or dynamically determined. A pre-determined amount of time may be set during manufacture of light panel 102 (e.g., may be 8 minutes, 4 minutes, etc.) and/or may be determined based on the intensity of the light (e.g., red light, infrared light) being emitted. For example, a user may set the timer while using light panel 102 at a higher light intensity setting. The timer may automatically be set for four minutes based on the higher light intensity setting. At a different time, the user may set the timer while using light panel 102 at a lower light intensity setting relative to the higher intensity setting. The timer may automatically be set for eight minutes based on the lower light intensity setting and may be controlled by an input receptor or an external control (e.g., an application with a GUI that receives input from a user.)

According to an implementation of the disclosed subject matter, light panel 102 may include or be operably coupled to one or more sensors, as further disclosed herein. The sensors may sense data that may be used to detect a health condition. The health condition may be any applicable condition including, but not limited to, inflammatory conditions, hair health, muscle condition, stress condition, sleep conditions, thyroid health, activity performance, or the like. The sensors may be include, but are not limited to a fitness tracker, visual sensor, ambient condition detection sensor, pH sensor, biochemical sensor, or the like. The sensors may be attached to a portion of light panel 102 or may be configured to communicate with light panel 102. For example, the sensor may be a wearable device that tracks a user's sleep and/or activity. The wearable device may communicate with light panel 102 in a wired or wireless manner or may communicate with a controller (e.g., a mobile device) that is in communication with light panel 102. Light panel 102 may be configured to adjust a setting based on the data obtained by the one or more sensors. For example, a sensor may collect data and provide the data to a machine learning model. The machine learning model may be trained to output one or more configurations of light panel 102 based on the input sensor data. The output may be based on detecting a given health condition or may be based on a health condition known to or provided to the machine learning model. For example, the sensor data may indicate a given health condition and the machine learning model may output one or more parameters to operate light panel 102 in order to treat and/or mitigate the expansion of the health condition. Alternatively, the machine learning model be a clinical decision support engine that may receive information from a cohort of users, clinical guidelines, health care provider or system, coach, physical therapist, care taker, or the like, (e.g., via a server, network, or other connection to the user's data). Based on the user's health information, the machine learning model may output parameters for operation of light panel 102 to treat or mitigate any conditions included in the health information. Alternatively, the machine learning model may receive inputs from the user. For example, the user may indicate thinning hair and that input may be provided to the machine learning model to provide outputs to optimize hair health.

The output may include a wavelength or set of wavelengths to output using light panel 102. The output may also or alternatively include duration data such that the timer can be dynamically set to turn light panel 102 off after the duration of time output by the machine learning model. Alternatively, the timer may be dynamically set to change a wavelength after a duration of time output by the machine learning model.

According to an implementation of the disclosed subject matter, settings for light panel 102 may be adjusted based on user or automation. As disclosed above, a machine learning model may output a configuration (e.g., intensity, wavelength, duration, etc.) based on sensor data and/or data received at the machine learning model. One or more settings of light panel 102 may be adjusted based on user input. A user may provide the user input directly via input receptors on light panel 102. Alternatively, light panel 102 may be connected to a user device (e.g., via a network, wired, or other wireless connection). The user may connect to light panel 102 via an application (e.g., a mobile device application, a website or web application, a standalone controller, etc.) and provide setting input via a graphical user interface (GUI) of the application.

Light panel 102 may be powered using a battery or via a direct power connection (e.g., via a wired or wireless power connection). A battery may be charged in any applicable manner such as a Universal Serial Bus (USB) charger, wireless (e.g., Qi) charger, magnetic connection charger, or the like. Light panel 102 may include an indication of a low battery directly on light panel 102 and/or may provide the indication via a user device (e.g., mobile phone) that light panel 102 is connected to.

Light panel 102 may include a plurality of dual light emitting diodes (LEDs) across or proximate to a surface of light panel 102. The dual LEDs may be or may include chips (e.g., a chip on board (COB) LED). The dual LEDs may be manufactured or placed on strips that are inserted or placed on or in light panel 102's housing. Alternatively or in addition, the dual LED s may be manufactured as a sheet that is applied to light panel 102. Alternatively, the dual LEDs may be individually placed in light panel 102. The dual LEDs may be equidistant from each other or may be spaced such that the outside edges have a higher distribution of dual LEDs and a central part has a lower distribution of LEDs, or vice versa. Alternatively, the dual LEDs may be arranged in any other pattern such as a circular pattern, random pattern, a design, or the like). As an example implementation, light panel 102 may include ten rows of dual LEDs with eight dual LEDs on each row. The rows may be staggered such that the electronics for the dual LEDs have sufficient space to not overlap or interfere with one other.

The irradiance of light panel 102 may be between approximately 100 mW/cm² and approximately 200 mW/cm². For example, the irradiance of light panel 102 may be approximately 150 mW/cm². The irradiance may be variable and/or based on one or more settings (e.g., as output by a machine learning model, set by a user, etc.).

Dual LED 104 may be configured to output light in both the red wavelength and light in the near infrared wavelength. The same dual LED chip may include a lens that outputs red wavelength light, near infrared wavelength light, or a combination of the same. Accordingly, light panel 102 may be configured to output either red light, infrared light, or a combination of the two using the same dual LED chips. In the example provided above including eighty dual LEDs (ten rows of eight dual LED chips) proximate to each other, each of the eighty dual LEDs may be configured to output red light, near infrared light, or a combination of the same.

According to implementations of the disclosed subject matter, light panel 102 may emit less than 50 V/m at 6 inches away or, more specifically may emit less than 50 V/m at 6 inches away. Such emission may be considered a safe amount of emission for use of light panel 102.

According to implementations of the disclosed subject matter, the dual LEDs 104 of light panel 102 may fluctuate at a frequency of 3 Hz or less. The fluctuation may be at a level that a human eye cannot perceive flickering of the dual LEDs 104 of light panel 102.

According to implementations of the disclosed subject matter, light panel 102 may incorporate security features. For example, light panel 102 may include a time used detection mechanism to detect how long a user has used light panel 102 and/or at what intensity light panel 102 was used for the duration. If the time used detection mechanism determines that light panel 102 was used for a duration greater than a recommended duration and/or at an intensity greater than a recommended intensity for a given duration, the time used detection mechanism may automatically shut off light panel 102 or reduce the intensity of light panel 102.

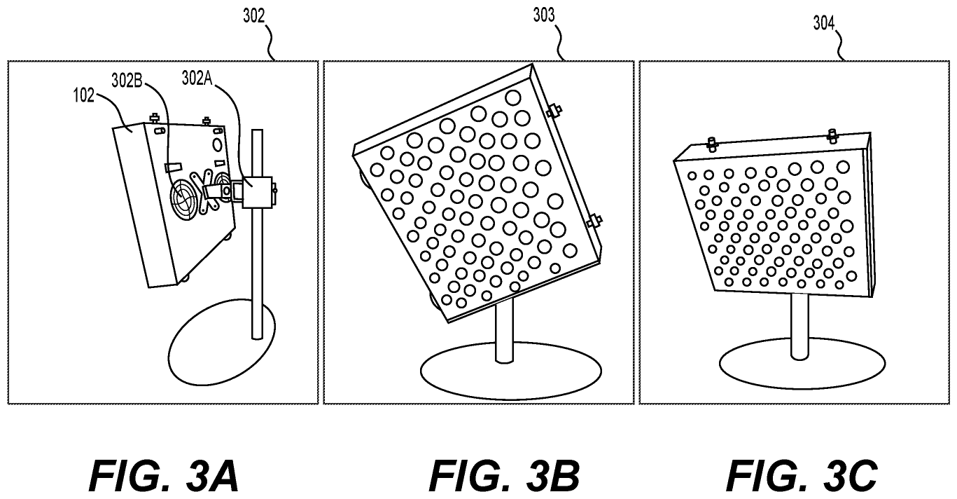

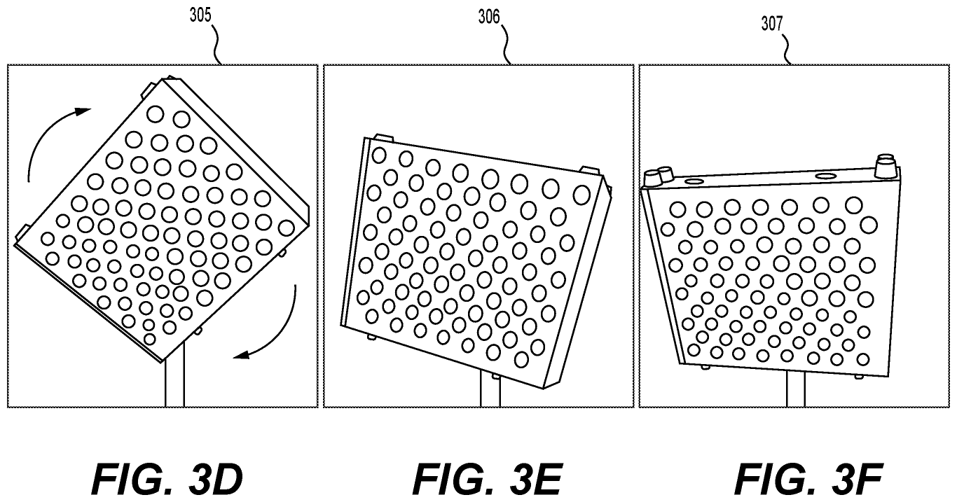

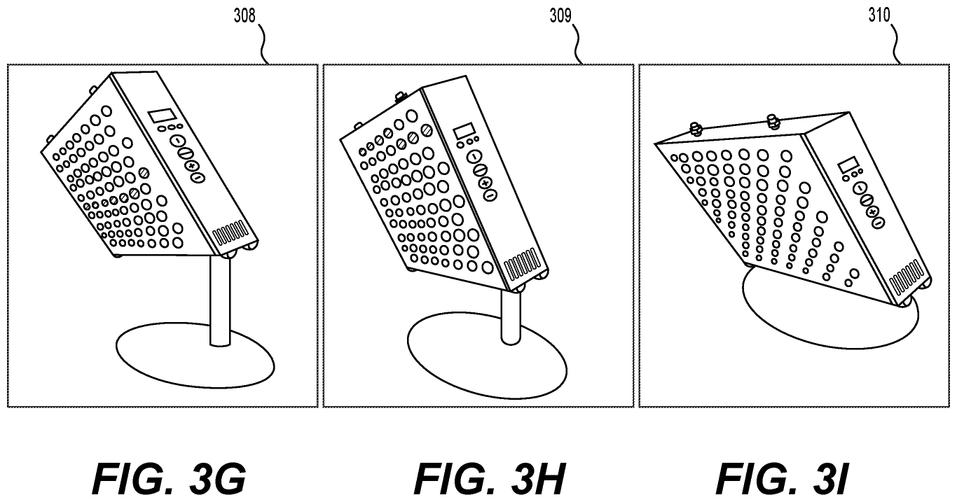

FIGS. 3A-3I provide a plurality of views 302-310 of light panel 102 on a stand, in accordance with implementations of the disclosed subject matter. FIG. 3A shows view 302, FIG. 3B shows view 303, FIG. 3C shows view 304, FIG. 3D shows view 305, FIG. 3E shows vie 306, FIG. 3F shows view 307, FIG. 3G shows view 308, FIG. 3H shows view 309, and FIG. 3I shows view 310. As shown in view 302, light panel 102 may be attached to a stand via a clasp 302A that holds light panel 102 at a given height on the stand. Light panel 102 may include a heat dissipation module 302B (e.g., a fan, a liquid heat transfer component, etc.) that manages temperature within light panel 102. As shown in views 304 and 308, light panel 102 may tilt forward or backwards. As shown in views 303, 305, 306, and 307, light panel 102 may be rotated in any applicable direction around clasp 302A. For example, light panel 102 may be arranged horizontally, vertically, or diagonally. As shown in views 304, 309, and 310, light panel 102's height may be adjusted higher or lower by using clasp 302A. It will be understood that one or more features (e.g., raising or lowering, tilting, rotating, etc.) may be done manually or automatically using a control. The control may be provided on light panel 102 or may be provided via a remote control or controller (e.g., a mobile phone).

FIG. 4 shows an example full body light panel 402. It will be understood that the disclosure provided herein for light panel 102 generally applies to full body light panel 402. FIG. 4 shows a height H2 for full body light panel 402. The height H2 may be, for example, approximately 36.2 inches. FIG. 4 also shows a width W2 for full body light panel 402. The width W2 may be, for example, approximately 11 inches. FIG. 4 shows a depth D2 for full body light panel 402. The depth D2 may be, for example, 2.6 inches. It will be understood that full body light panel 402 may be of variable heights, widths, and depths, other than those disclosed herein.

Full body light panel 402 may include, for example, approximately forty rows of eight dual LEDs 104 each (i.e., approximately 320 dual LEDs 104). Full body light panel 402 may be configured to provide red light (e.g., with a wavelength of approximately 630 nm, or a range of approximately 600 nm-750 nm), near infrared light (e.g., with a wavelength of approximately 850 nm, or a range of approximately 750-1000 nm), or a combination thereof. Generally, full body light panel 402 may be configured to provide light in the range of approximately 600 nm-1000 nm. For example, FIG. 4 shows full body light panel 402 emitting red light at approximately 660 nm at 404, near infrared light at approximately 850 nm at 406, and a combination of red light at approximately 660 nm and near infrared light approximately 850 nm at 408. It will be understood that when emitting either red light or near infrared light, each of the dual LEDs 104 may emit the respective frequency of lights (e.g., not a subset of the dual LEDs 104).

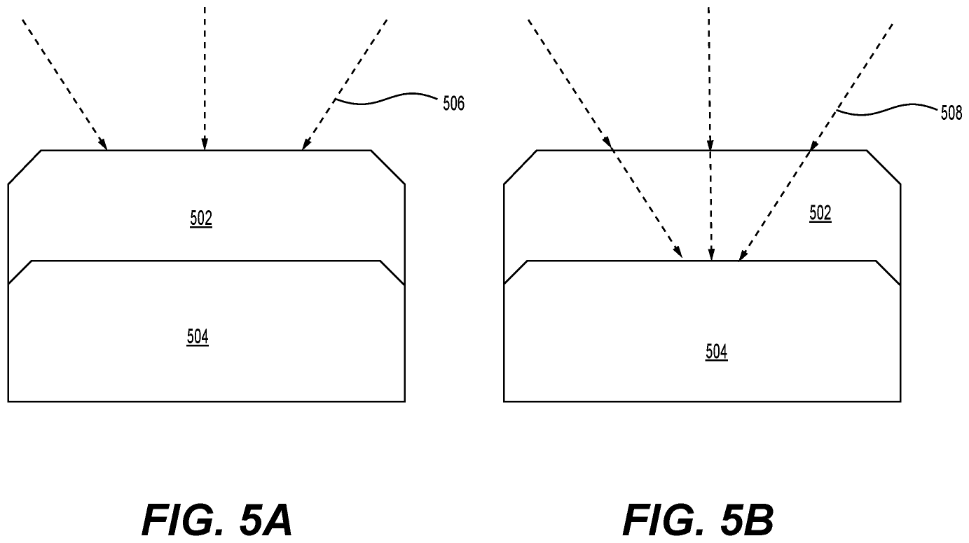

Dual LEDs 104 may be each be configured to emit light (e.g., red light) that can interact with cells on a surface as well as light (e.g., near infrared light) that can interact with cells deeper than a surface. FIGS. 5A and 5B depict diagrams representing a surface 502 (e.g., a user's skin, hair or head, etc.) and a sub-surface area 504 (e.g., tissue, scalp, muscles, etc.). As disclosed herein, light panel 102 may include dual LED 104 each configured to emit red light, near infrared light, and/or a combination of the two. The ability to emit red light, near infrared light, and/or a combination of the two may result in light panel 102 efficiently reducing inflammation, increasing circulation, and/or optimizing functionality of mitochondria (e.g., for a user of light panel 102). As shown in FIG. 5A, red light 506 may emit from light panel 102 and be incident on the surface 502. The red light 506 may interact with the surface 502 to reduce inflammation, increase circulation, and/or optimize functionality of mitochondria, or the like, at the upper surface 502. Additionally, as shown in FIG. 5B, near infrared light 508 may interact with the sub-surface area 504 to reduce inflammation, increase circulation, and/or optimize functionality of mitochondria, or the like, at the sub-surface area 504. Accordingly, using dual LEDs 104, light panel 102 may have an effect on both the surface and sub-surface tissue that it interacts with.

The dual LED circuits configured to emit red, near infrared, or a combination, as disclosed herein (e.g., dual LEDs 104), may be applied in alternate light panels and/or configurations than those disclosed above. For example, one or more LEDs for red light or near infrared light may be provided as patches that a user may place on her body (LED Patches). The LED patches may include a one time or a rechargeable battery. The battery may be rechargeable while a user has the patch attached to her body. The LED patches may include an adhesive element such that an LED patch may adhere to a portion of the user's body using the adhesive element. Alternatively, the LED patches may include a band, clasp, or other retaining element that maintains an LED patch proximate to a user's body (LED body wrap). An LED body wrap may be worn around, for example, a shoulder, a knee, hips, an ankle, an elbow, a wrist, an abdomen, a neck, or any other applicable portion of the body.

As further discussed herein, a light integrated device may be a light panel (e.g., light panel 102, light panel 110, light panel 402, etc.), a shower head, an exercise equipment, a bulb, a transcranial device, a headwear, a mask, an eyewear device, a wearable device, an intra-body device, or the like. A light integrated device may include one or more one or more dual LEDs 104, as disclosed herein.

According to an implementation, one or more dual LEDs 104 may be installed into or attached to a shower head (LED shower head). The LED shower head may be activated when the shower is turned on or may be activated independent of the shower. For example, a user may use the LED shower head prior to or after turning on the water portion of the shower. The settings on the LED shower head may be configured to, for example, target hair loss or scalp health. The LED shower may be configured to adjust its settings based on whether water is running or not running in the shower. For example, if water is running in the shower, the intensity of the red and/or near infrared light may be increased to accommodate for distortion due to the water.

According to an implementation, one or more dual LEDs 104 may be installed into or attached to exercise equipment such as stationary bikes, treadmills, automated stairs, squat racks, multi-item weight machines, or the like (LED exercise equipment). The LED exercise equipment may be configured to activate one or more dual LEDs 104 based on the location of an individual, the exertion level of the individual, or the like. For example, a stationary bike may activate one or more dual LEDs 104 directed at a user using the stationary bike. The light emission, intensity, and/or duration of the one or more dual LEDs 104 may be adjusted based on the level of exertion of the user, as recorded by a suitable sensor (as described above) or operably coupled to the stationary bike.

According to an implementation, one or more dual LEDs 104 may be installed as or attached to a bulb. The LED bulb may be connected to a power source or may be connected to researchable battery. As an example, an LED bulb for emitting red and near infrared light may be placed in a hyperbaric oxygen therapy chamber (HBOT) such that a user may receive light therapy using the LED bulb as well as oxygen therapy at the same time. The LED bulb may be configured to operate in concert with oxygen therapy. For example, one or more sensors may detect biometric data for a user in the HBOT chamber. The LED bulb may adjust light emission, wavelength, and/or duration based on the changes registered by the one or more sensors. Similarly, one or more dual LEDs 104 may be placed in or around a mirror (e.g., a vanity mirror). A sensor may detect the presence of a user in front of the mirror (e.g., while brushing teeth) and may activate and/or operate the one or more dual LEDs 104 accordingly. As another example, one or more dual LEDs 104 may be included in vehicles (e.g., in headrests or as inserts in occupant cabin roofs), tanning beds, pools, Jacuzzis, toilets, saunas, or the like. The dual LEDs 104 may be configured to adjust wavelength, intensity, and/or duration based any medium saturation (e.g., humidity, water, etc.). Safety considerations (e.g., in vehicles) may be considered such that, for example, a sensor may detect the amount of ambient light and may only activate dual LEDs 104 if the ambient light indicates daylight. Using this configuration, a vehicle may not emit red or near infrared light during night hours, in accordance with safety regulations.

According to an implementation, one or more dual LEDs 104 may be incorporated into a transcranial device (e.g., transcranial LEDs). For example, transcranial LEDs may be configured to provide red and near infrared emission to areas of the brain. The transcranial LEDs may be operated remotely and/or may be paired with a sensor to detect optimal locations for light emission.

According to an implementation, one or more dual LEDs 104 may be placed in headwear such as a helmet type device such that light emission is directed to a user's hair and/or scalp (helmet LED). The helmet LED may be configured to adjust emission to target areas of the scalp based on a quantity of the hair. For example, an area of a scalp may receive a wavelength emission at an intensity and for a duration based on the amount of hair. As another example, light emission may be adjusted to increase the probability of reaching a scalp through hair.

According to an implementation, one or more dual LEDs 104 may be included in a mask (LED mask). The LED mask may powered by a battery or, for example, may be powered by airflow or air differentials achieved as a result of breathing. The LED mask may also reach the inside of a user's mouth to provide red and near infrared light therapy inside the user's mouth.

According to an implementation, one or more dual LEDs 104 may be configured to promote eye health (eyewear device). An eyewear device may be, for example, incorporated into eyewear such as, but not limited to, eyeglasses, eyepatches, sunglasses, and/or headwear such as, but not limited to, hats, visors, or the like. The eyewear device may be configured to emit red and near infrared light that is suitable for a user's eyes. The eyewear device may include safety features, as those disclosed herein, to mitigate risk to a user's eye.

According to an implementation, one or more dual LEDs 104 may be provided in wearable items (wearable LED). A wearable LED may be incorporated into clothing, bandages, shoes, sandals, undergarments, bedsheets, or the like. For example, wearable LEDs may be incorporated into a blanket such that a user using the blanket is immersed in red and near infrared light under the blanket. The blanket may be configured to emit a low irradiance amount of light such that it can be used for long durations (e.g., overnight). Alternatively, the blanket may be configured to activate for limited short bursts (e.g., when a user first starts using the blanket, based on an alarm or wake up time, etc.). The short bursts may be, for example, 20 minutes. The blanket may function as a heated blanket such that heat generated by the wearable LEDs is dissipated to the user. A user may be able to control the light and/or heat settings using an application GUI, as discussed herein. As another example, wearable LEDs may be placed inside clothing (e.g., a sleeve, a body suit, or the like). Such a clothing-based wearable LED arrangement may target injury or general recovery in addition to other health conditions.

According to an implementation, one or more dual LEDs 104 may be provided in intra-body forms (intra-body LED). For example, one or more LEDs may be swallowed as a capsule, may be implanted, or may be an intravascular insertion. The intra-body LED may activate using a remote device, may activate based on sensor input, and/or may activate based on a given amount of degradation of material surrounding the intra-body LED. For example, an intra-body LED may be swallowed as a capsule. The capsule may be surrounded by digestible material that may be digested in a user's gastroenterology (GI) tract. Upon digestion, the intra-body LED inside the digestible material may be exposed and the GI tract or portions thereof may be exposed to red and near infrared light. The intra-body LED may be configured to dissolve inside a user's body or may be removed from the user's body. The intra-body LED or another implementation of dual LEDs 104 may be used to treat, mitigate, and/or prevent autoimmune diseases.

According to an implementation, one or more dual LEDs 104 may be configured for animal use (e.g., pet use). Such dual LEDs 104 may be placed in beds, collars, chew toys, leashes, or the like.

According to an implementation, consumable products (e.g., food, water, etc.) may be infused with red and near infrared light from one or more dual LEDs 104. The wavelength, intensity, and/or duration may be adjusted based on the type of consumable (e.g., based on how much light enters the food for an appropriate amount of infusion).

According to an implementation, a light panel may be configured for plant immersion such that they may be placed such that indoor and/or outdoor plants receive red and near infrared light. One or more sensors that detect properties of the plants and light panel may be adjusted accordingly, as disclosed herein.

According to an implementation, one or more dual LEDs 104 and/or a light panel may be configured to be provided in space (e.g., for use by an astronaut). The dual LEDs 104 and/or a light panel may be configured to operate in a vacuum or spaceship conditions. Such configuration may include pressure-based components, gravity-based components, or the like. Additionally, the dual LEDs 104 and/or a light panel may be configured to emit light in a vacuum or spaceship environment. The dual LEDs 104 and/or a light panel may further be configured to emit light that is incident on a user within a spacesuit.

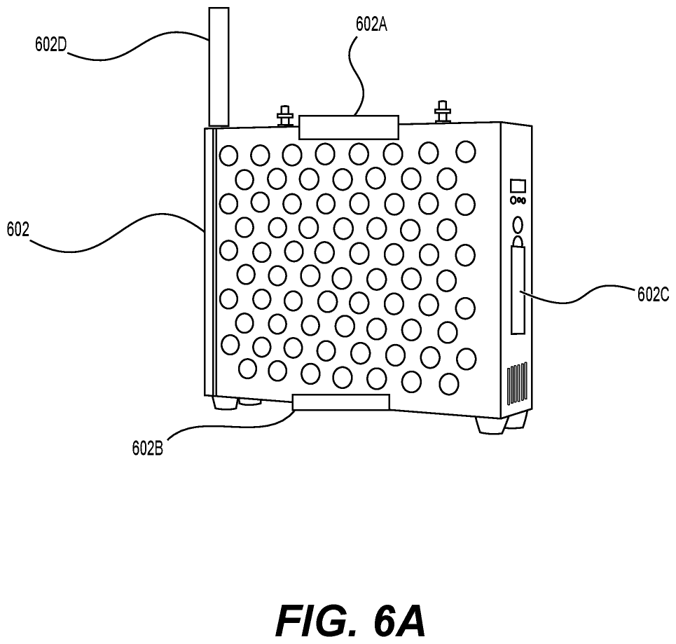

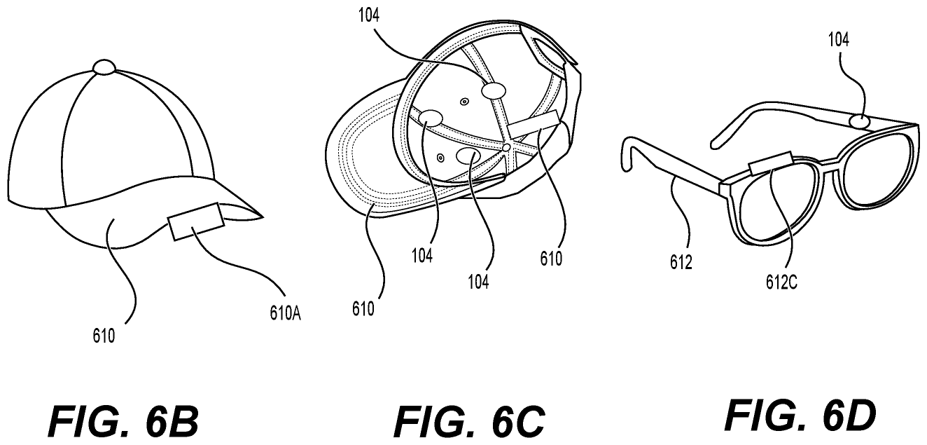

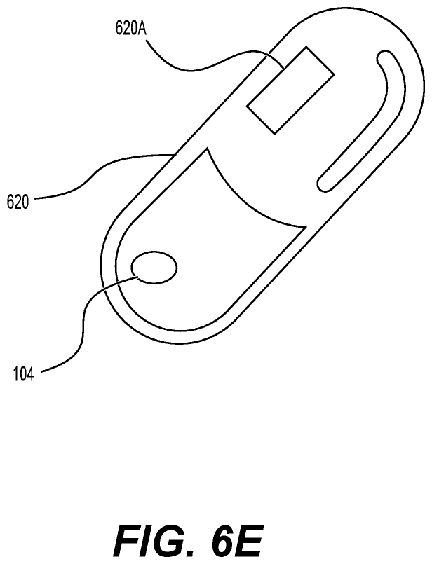

According to implementations of the disclosed subject matter, a light integrated device may include one or more sensors. FIG. 6A shows a light panel 602 with sensors 602A, 6026, 602C, and 602D. Sensor 602A and 602B may be positioned on a surface of light panel 602 that emits light (e.g., towards a user using light panel 602). Sensors 602A may be configured to detect user attributes (e.g., user temperature, complexion, movement, gaze, features, biometric data, etc.). Sensor 602C may be positioned on a side of light panel 602. Sensor 602D may extend out from light panel 602. Sensors 602C and 602D may be configured to detect external properties (e.g., ambient properties, device properties, etc.) such as, but not limited to ambient temperature, device temperature, light conditions, etc.

FIG. 6B shows head gear 610 including a sensor 610A. Sensor 610A may be positioned such that it can sense data associated with a user wearing head gear 610. Sensor 610 may be configured to detect user attributes and/or external properties as disclosed herein. FIG. 6C shows a bottom view if head gear 610, sensor 610B, and a plurality of dual LEDs 104. Sensor 610B may be configured to detect user attributes and/or external properties, as disclosed herein. For example, sensor 610B may be configured to detect a hair volume of a user wearing head gear 610.

FIG. 6D shows eyewear 612 including a sensor 612C and a dual LED 104. Sensor 612C may be configured to detect user attributes and/or external properties, as disclosed herein.

FIG. 6E shows a capsule 620 including a sensor 620A and a dual LED 104. Sensor 620A and/or dual LED 104 of FIG. 6E may be internal or external to capsule 620. Sensor 620A may be configured to detect user attributes, as disclosed herein. For example, capsule 620 may be ingested by a user and sensor 620A may transmit sensed data related to the user to an external component.

It will be understood that the sensors and configurations shown in FIGS. 6A-6E are examples only. One or more sensors and/or dual LEDs 104 may be attached to, integrated within, or otherwise associated with any applicable light integrated device. For example, one or more sensors and/or dual LEDs 104 may be attached to, integrated within, or otherwise associated with patches, shower heads, exercise equipment, bulbs, transcranial device, head gear, masks, glasses, wearable devices or garments, or the like. It will also be understood that one or more sensors may be stand alone sensors or sensors associated with devices that communicate with a component (e.g., a processor). Such stand-alone or device-based sensors may sense sensed data that is used to configure one or more light integrated devices, as further discussed herein.

The sensors shown in FIGS. 6A-6E or otherwise discussed herein may be one or more of a visual sensor, chips, laser sensor, temperature sensor, ambient condition detection sensor, pH sensor, biochemical sensor, motion sensor, material sensor, or the like. The sensors shown in FIGS. 6A-6E or otherwise discussed herein may be configured to sense sensed data which may include, but is not limited to, data related to: biometrics, movement, chemicals, shapes, objects, electrical signals, body data, mitochondria, proteins, glucose, lactate, urea, uric acid, microorganisms, serum, blood, and/or the like. The sensed data may be used to detect a medical condition or a treatment.

The sensors shown in FIGS. 6A-6E or otherwise discussed herein may be powered using the same power source (e.g., a battery) as a light integrated device. Alternatively, or in addition, the sensors may be powered using an external energy source (e.g., a power source (e.g., electrical energy), kinetic energy, heat energy, etc.).

The sensors shown in FIGS. 6A-6E or otherwise discussed herein may be activated continuously or based on one or more triggers. A sensor activation may correspond to sensing of given sensed data by a given sensor. A sensor activation may include receiving an input (e.g., a physical input, a biochemical input, an electrical input, a motion input, etc.) and/or may include converting an input to a sensed signal. The sensed signal may be an electrical signal, a change of a state, a change of a property, or the like. For example, a sensor may be configured to detect user temperatures. The sensor may continuously sense temperatures such that a temperature detection mechanism constantly receives a signal (e.g., a temperature signal) and may provide the signal to a processor configured to generate a temperature based on the signal.

A trigger-based sensor activation may be based on a temporal trigger (e.g., a time, a duration of time, a chronic time, etc.), or an event-based trigger. An event-based trigger may be a trigger that is activated upon the occurrence of a given event. An event-based trigger may be based on a signal from a sensor. For example, a pressure sensor or group of sensors may determine that a light integrated device meets one or more pressure criteria such as when a headwear device is pressed against a user's head as the user wears the headwear device. The pressure sensor or group of sensors may detect that the detected pressure meets one or more thresholds indicating a user wearing the headwear device. Accordingly, the pressure sensor or group of sensors may emit a signal indicating that the headwear is worn by the user. Based on the signal, one or more other sensors may be activated, where the event in this case is the pressure sensor or group of sensors emitting the signal indicating that the headwear device is worn by the user.

An event-based trigger may be based on an output determined based on user information, sensed information, or the like. User information may be a user history, a current user state, or the like. A current user state may be received from one or more components such as external sensors, a database, or the like (e.g., a blood pressure device, a health care database, etc.). The user information and/or sensed information (e.g., sensed by one or more sensors associated with a light integrated device or an external sensor) may be input at a machine learning model and the machine learning model may determine when to trigger a sensor activation based on the user information and/or sensed information.

According to an implementation of the disclosed subject matter, one or more sensors (e.g., integrated sensors or external sensors) associated with a light integrated device may be infra-red or red sensors. Such sensors may capture high signal-to-noise and high-resolution photoplethysmography (PPG) measurements from the surface or sub-surface of a user's skin (e.g., up to approximately 20 mm or up to approximately 10×deeper than green light) to extract biometric sensed data. For example, light panel integrated infra-red or red sensor may project light towards a user using the light panel. The response from the projected light may be sensed data that can be used to identify a medical condition or medical status of a user.

According to an implementation of the disclosed subject matter, one or more sensors (e.g., integrated sensors or external sensors) associated with a light integrated device may be laser sensors. Such sensors may use one or more lasers to detect user properties. For example, laser sensors and the corresponding sensed data may be used for detection of skin conditions, eye conditions, hair density, or the like. The laser sensors may generate a digital readout, which may be used to determine a condition.

According to an implementation of the disclosed subject matter, one or more sensors (e.g., integrated sensors or external sensors) associated with a light integrated device may be ambient or device sensors. An ambient sensor may detect ambient conditions (e.g., temperature, humidity, light, etc.). A device sensor may detect device conditions for the light integrated device (e.g., device temperature, component variance or drift, device power, etc.).

According to an implementation of the disclosed subject matter, one or more sensors (e.g., integrated sensors or external sensors) associated with a light integrated device may sense biochemical markers in bio fluids, such as sweat, tears, saliva and/or interstitial fluid. For example, an oral device (e.g., a mouthguard, a retainer, etc.), a user device (e.g., a hand-held device, a patch, etc.) or the like may include one or more sensors. Such sensors may be non-invasive and may include one or more electrochemical and/or optical biosensors. Data sensed using such sensors may be used to identify or determine information related biomarkers including metabolites, bacteria, and hormones. For example, saliva may be collected by or near a sensor in communication with the light integrated device. The saliva may be in contact with a non-invasive electrochemical sensor configured to detect one or more biomarkers including bacteria from the saliva. Concentration of certain biochemical markers in saliva may be highly relevant to those in blood, as a result of exchange between salivary glands and blood. Accordingly, as further discussed herein, sensed data (e.g., sensed salvia) may be used to determine or predict blood properties.

The non-invasive electrochemical sensor may sense electrochemical attributes of the saliva and may generate electrical signals based on the same. The electrical signals may be received at a processor located at or remote from the light integrated device. The processor may convert the electrical signals to data that represents the presence of one or more biomarkers (e.g., a type, quantity, quality, etc., of bacteria). Alternatively, the non-invasive electrochemical sensor may itself be configured to output such data.

According to an implementation of the disclosed subject matter, one or more sensors (e.g., integrated sensors or external sensors) associated with a light integrated device may sense exhaled breath condensate (EBC) content. EBC content may include, but is not limited to, mediators including adenosine, ammonia, hydrogen peroxide, isoprostanes, leukotrienes, nitrogen oxides, peptides and cytokines. Concentrations of these mediators are influenced by lung diseases and modulated by therapeutic interventions. Similarly, such one or more sensors may detect pH levels based on collected EBC. As further discussed herein, properties of EBC content and/or changes in the same may indicate the presence or probability of conditions (e.g., respiratory conditions).

According to an implementation of the disclosed subject matter, one or more sensors (e.g., integrated sensors or external sensors) associated with a light integrated device may sense volatile organic compound (VOC) biomarkers. VOC biomarkers may be indicative of environmental exposures such as that caused by particulate matter from burn pits, oil field fires, metal alloys, or the like.

According to an implementation of the disclosed subject matter, one or more sensors (e.g., integrated sensors or external sensors) associated with a light integrated device may be pressure sensors, force sensors, or movement sensors for monitoring the body of a user. Such sensors may monitor user movement, user body part properties (e.g., muscle properties, range of motion, etc.) Such sensors may measure the force applied by orthopedic devices, supportive devices, by a user or the like. Such sensors may capture changes a user's body part or parts (e.g., muscles, stature, skin, etc.) over time. Such sensors may be implemented using a force plate, weight sensor, or other component configured to detect force-moment (e.g., six force moment) of wires, bands, and/or brackets at one or more body parts. One or more stress sensors may be integrated on a chip via complementary metal oxide semiconductor (CMOS) technology. The chip may embedded into the light integrated device or other component in communication with the light integrated device. Based on the measured data, force-moment detection be determined. The data may be applied to or using one or more simulations. For example, isolated calibration loads may be complemented by using finite element (FE) simulations.

According to an implementation of the disclosed subject matter, one or more sensors (e.g., integrated sensors or external sensors) associated with a light integrated device may be temperature sensors. The temperature sensors may be used, for example, for changes in a user's overall body temperature, a body part temperature, or the like during normal conditions or during an activity (e.g., exercise, sleep, etc.). For example, temperature may be a targeting parameter of inflammation and the local temperature near a body part or implant may be used as an indicator to monitor medical conditions. According to an example, a multi-channel temperature sensors may be microfabricated based on a photo-definable polyimide. Temperature sensors may output temperatures (e.g., over time) to detect temperature changes and/or medical conditions.

According to an implementation of the disclosed subject matter, one or more sensors (e.g., integrated sensors or external sensors) associated with a light integrated device may be chemical sensors. Such sensors may sense body or body fluid properties. Such sensors may include soft, low-profile, intrabody electronics configured for continuous real-time monitoring of chemical properties and/or chemicals (e.g., sodium concentrate, proteins, anti-bodies, etc.). Such sensors may include sodium ion-selective sodium electrodes (ISEs), made of polymers with high selectivity, wide signal range, and rapid response time, selected for monitoring sodium levels in, for example, saliva.

According to an implementation of the disclosed subject matter, one or more sensors (e.g., integrated sensors or external sensors) associated with a light integrated device may be physical sensors. Physical sensors may capture the motion of user activity (e.g., overnight while a user wears or is proximate to a light integrated device or other device in communication with a light integrated device). A user's body or body part activity may be sensed by such sensors and sensed data may be provided to a processor, as discussed herein. The user's body or body part activity may be provided as an input to a machine learning model which may generate a machine learning output, as further discussed herein. The machine learning output may be based on comparing the activity to known activity associated with known conditions (e.g., tremors) to identify a medical condition.

According to an implementation of the disclosed subject matter, one or more sensors (e.g., integrated sensors or external sensors) associated with a light integrated device may be optical sensors. Such sensors may use light-based techniques to detect user's body properties such as pigmentation properties, chemical properties, blood or blood flow properties, or the like. Such sensors may use a light-based techniques to quantify magnetic fields produced by neurons firing in the brain and may be used instead of magnetic resonance imaging (MRI) machines to create similar imaging, eliminating. Additionally, according to an implementation, the light integrated device or associated device may include components (e.g., copper, galvanized steel, aluminum, etc.) that provide expensive cooling or electromagnetic shielding required when undergoes an MRI scan.

According to an implementation of the disclosed subject matter, one or more sensors (e.g., integrated sensors or external sensors) associated with a light integrated device may be ultraviolet (UV) sensors. The UV sensors may use UV light to generate sensed data. According to an implementation, a user may apply or consume a fluorescent solution. The light integrated device or associated device may be activated or positioned such that the UV sensors can detect user properties (e.g., chemical makeup, plaque, etc.) based on reminisce of the fluorescent solution. It will be understood that the fluorescent solution may not be required for the UV sensors to detect user properties, though such a solution may improve the detectability of the same.

According to an implementation of the disclosed subject matter, one or more sensors (e.g., integrated sensors or external sensors) associated with a light integrated device may be biosensors. Biosensors may be used to generate sense sensed data (e.g., based on sensed signals) that allows assessment of health and disease states. The biosensors may generate signals based on fluids, cells, microorganisms, etc., as well as other compounds that may be found in or pass in or about a user's body.

According to an implementation of the disclosed subject matter, one or more sensors (e.g., integrated sensors or external sensors) associated with a light integrated device may be glucose sensors. Such sensors may provide continuous glucose monitoring (CGM) based on user body properties such as blood properties or saliva properties. Such sensors may be configured to detect blood glucose or sense markers indicative of blood glucose. Such sensors may be configured to detect ketones and/or ketone properties which may be used to determine glucose levels. According to an implementation, such sensors may analyze blood and may detect blood glucose levels.

According to an implementation of the disclosed subject matter, one or more sensors (e.g., integrated sensors or external sensors) associated with a light integrated device may be mitochondrial sensors. Mitochondrial sensors may be configured for quantum sensing such as by using one or more quantum objects. A quantum object may be the unpaired electron associated with a nitrogen-vacancy (NV) center in diamond which can be exploited as an extraordinarily sensitive room temperature magnetometer, deployed for nanoscale temperature measurements. Such objects may be used to detect temperature changes, as discussed herein.

Mitochondrial sensors may include a PTEN-induced kinase 1 (PINK1) sensor. PINK1 is a serine/threonine protein kinase which localizes to a mitochondrion and regulates its function and turnover by sensing when the mitochondrion is damaged. PINK1 may be used for mitochondrial health by facilitating fusion and fission, mitophagy, and mitochondrial transport pathways, which serve as a quality control system to remove dysfunctional or damaged mitochondrion from the cell. Accordingly, mitochondrial sensors may detect the presence, quality, and/or quantity of PINK1.

Mitochondrial sensors may be configured to detect fluorescence-based assays including measurements of mitochondrial calcium, superoxide, mitochondrial permeability transition, and membrane potential.

According to an implementation of the disclosed subject matter, one or more sensors (e.g., integrated sensors or external sensors) associated with a light integrated device may be used to mimic blood tests. Such an implementation may use one or more of short-wavelength infrared sensors, semiconductor photonics and/or electrooptic sensors, laser printed graphene (LIG) based electrode biosensors, or the like. A short-wavelength infrared sensor may be used to detect the amount of sugar in a user's blood. A semiconductor photonics and/or electrooptic sensor may be configured to detect levels of glucose, lactate, urea, serum albumin, and/or other substances in a user's blood. LIG sensors may combine high electrical conductivity of graphene with an ultra-easy fabrication procedure that simply requires a CO2 laser printer. LIG sensors may be implemented a high porosity and an interlocking design to enhance the biosensor's sensitivity. Data output by such sensors may be used to generate results similar to a blood test.

According to implementations of the disclosed subject matter, a light integrated device or an associated component may be configured to sense signals to generate sensed data using one or more of the sensors disclosed herein. The sensed data may be processed by a processor (e.g., an internal or external processor). A machine learning model may be used to generate a machine learning output. The machine learning output may include an indication, signal, instruction, or the like to configure the light integrated device to output light at a wavelength, intensity, rate, duration, and/or frequency. The configuration of the light integrated device may be, for example, to treat or otherwise improve a condition indicated based on the sensed data.

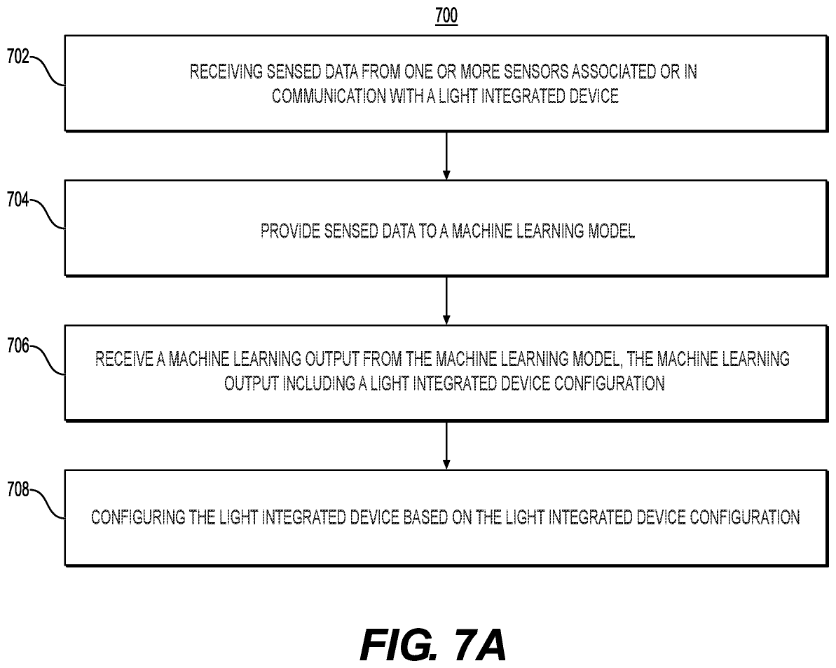

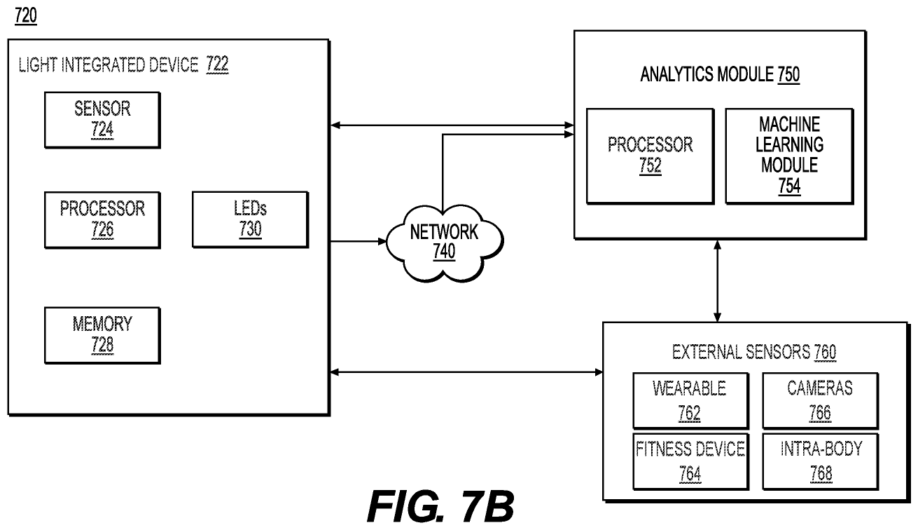

FIG. 7A depicts a flow chart 700 for targeted light therapy. At 702, sensed data from one or more sensors of the light integrated device, or a device in communication with the light integrated device, may be received. The sensed data may be any of the sensed data discussed herein generated by one or more of the sensors discussed herein. The sensed data may be generated by the one or more sensors continuously or based on an event, as discussed herein. The sensed data may be received an internal or external processor, as further discussed herein in reference to FIG. 7B. The sensed data may be sensed by one or more sensors in a first format (e.g., a sensor specific format, a signal, or the like) and may be converted into a second format (e.g., by a processor).

At 704, the sensed data may be provided to a machine learning model. The machine learning model may be trained based to generate a machine learning output based on sensed data. The machine learning model may be trained based on medical conditions, historical changes that effect medical conditions, light properties, or the like. The machine learning model may be trained by adjusting one or more of weights, layers, biases, nodes, or the like to correlate sensed data to medical conditions such that the correlation may be a probability or likelihood (e.g., above a respective threshold) that sensed data indicates the presence or likelihood of a medical condition. For example, the machine learning model may receive the sensed data and may apply the sensed data to one or more of weights, layers, biases, nodes, or the like to determine if the sensed data indicates the presence or likelihood of a given medical condition. Alternatively, for example, the machine learning model may determine health properties of a user based on the sensed data. The health properties may be indicative of one or more medical conditions. The machine learning model may generate a machine learning output based on the correlation.

According to implementations of the disclosed subject matter, the machine learning model may be a single machine learning model or may include multiple machine learning models. For example, sensed data from different sensors may be provided to respective machine learning models and a central machine learning model configured to receive outputs from each of a plurality of respective machine learning models may generate the machine learning output.

According to implementations of the disclosed subject matter, the machine learning model may be trained and/or updated based on cohort data. Cohort data may correspond to sensed data and/or outcomes for a cohort of users. The cohort of users may be any group of users (e.g., other users that use similar light integrated devices, users with medical conditions, users that received light therapy for medical conditions, results of light therapy for users, or the like). For example, cohort data may include actual or simulated sensed data for the cohort of users. The cohort data may further include light therapy or other treatments implemented for the cohort of users and what effect the light therapy or other treatments had for the cohort of users. Light therapy treatments or other treatments correlated to light therapy treatments that had a positive effect for the cohort of users may be weighted heavily when training the machine learning model to generate a machine learning output. Accordingly, for example, sensed data for a given user may be compared to sensed data for the cohort of users. The machine learning model may apply greater weight to the layers, biases, weights, nodes, etc. trained based on the cohort of users that match the sensed data for a given user based on a matching threshold. Further, the machine learning model may apply greater weight to light therapy that had a positive effect for those matched cohort of users, when generating a machine learning output. Positive outcomes may include a reduction in presence or intensity of a given medical condition, the treatment of a medical condition, the prevention of a medical condition, or the like, for one or more medical conditions indicated by the sensed data.

At 706, a machine learning output may be received from the machine learning model. The machine learning output may include a light integrated device configuration. Accordingly, the machine learning output may include a configuration that may be best indicated by the machine learning model to treat, mitigate, and/or prevent a condition (e.g., a medical condition) for a given user. The light integrated device configuration may include a dual LED setting.

A light integrated device configuration may include one or more parameters of wavelengths of light, intensity of light, rate (e.g., pulse rate), duration of treatment, frequency of treatment, or the like. The wavelength(s) of light may correspond to the wavelengths that the light integrated device is configured to output based on one or more other parameters. The machine learning output configuration may indicate how the light integrated device outputs one or different wavelengths at different intensities, a given rate or different rates of output of the wavelengths, a duration or durations of time for output of the wavelengths, frequencies of output of the wavelengths, or the like. For example, the configuration may indicate that a first wavelength should be output at a first intensity for five minutes, at a second intensity for seven minutes. The configuration may further indicate that after the twelve total minutes of outputting the first wavelength, a four minute break where no wavelength is output should be implemented. The configuration may further indicate that after the break, a second wavelength should be output at a third intensity for two minutes, at a second intensity for nine minutes. The configuration may further indicate that the previous steps should be cycled through four times before the light integrated device automatically shuts off.

The dual LED setting may include one or more outputs using one or more dual LEDs such that the one or more outputs include activation of a dual LED based on at least two different wavelengths. Accordingly, the dual LED setting may include an output of a wavelength that is a combination of at least two different wavelengths. Alternatively, or in addition, the dual LED setting may include outputting a first wavelength (e.g., red light) at a first time and a second wavelength (e.g., near red light) at a second time. Alternatively, or in addition, the dual LED setting may include outputting a first wavelength at a first intensity and outputting a second wavelength at a second intensity. Alternatively, or in addition, the dual LED setting may include outputting a first wavelength for a first duration and outputting a second wavelength for a second duration. The dual LED setting may be a part of the light integrated device configuration output by a machine learning model, as discussed herein.

According to an implementation, sensed data generated based on a first sensor or group of sensors may be used to generate a machine learning output. Subsequently, sensed data from a second sensor or group of sensors may be used to update the machine learning output. The sensed data from the second sensor or group of sensors may be generated based on a first machine learning output configuration indicating a request for sensor data from the second sensor or group of sensors. For example, the machine learning model may determine that sensed data from a first sensor or group of sensors is not sufficient (e.g., in quantity, quality, type of data, etc.). Accordingly, the machine learning output may indicate a request for additional data from the second sensor or group of sensors. According to an implementation, the second sensor or group of sensors may include the first sensor or group of sensors (e.g., if additional data from the same sensors is requested).

At 708, the light integrated device may be configured based on the light integrated device configuration indicated the machine learning output. The light integrated device may be configured using a processor, as further discussed herein. The configuration indicated by the machine learning output may be implemented until an updated configuration is received, or until the light integrated device is reset using a reset signal (e.g., provided by a processor or via user input).

The intensity of light output by the light integrated device may be adjusted by adjusting the power provided to one or more LEDs. Alternatively, or in addition, the intensity may be adjusted by a signal configured to increase or decrease the intensity output by one or more LEDs.

The wavelength output by the light integrated device may be adjusted by activating and/or deactivating one or more LEDs. Alternatively, or in addition, the wavelength may be adjusted by modifying a property of the one or more LEDs. For example, each LED may have an on-board chip configured to modify the wavelength output by a given LED. A dual LED may include multiple bulbs configured to output one or more wavelengths and a wavelength may be adjusted by activating respective bulbs by providing a single to the on-board chip.

According to an implementation of the disclosed subject matter, updated sensed data may be provided to the machine learning model. The machine learning model may generate an updated machine learning output based on the updated sensed data. The updated machine learning output may be adjusted based on a current configuration (e.g., a previous machine learning output). The updated machine learning output may be adjusted based on updated cohort data that may also be received at the machine learning model. Accordingly, the light integrated device may be continuously configured, at least in part based on changes effected by light therapy from previous configurations and/or changes that a given user undergoes (e.g., health changes, diet changes, medication changes, etc.).

According to an implementation, the machine learning model may receive external input. The external input may be from one or more sensors external to the light integrated device and/or user data. User data may be user diet information, user medication information, user health information, user activity information, or the like. For example, user diet information may be input by a user or an automated system. The user diet information may be used to determine a light integrated device configuration such that, for example, a change to a salty diet may require changing a light (e.g., wavelength) output by the light integrated device.

According to an implementation, the machine learning output may include an external device configuration. For example, the machine learning model may detect a glucose level of a given user. The glucose level may indicate the requirement of additional insulin at a given time. Accordingly, the machine learning output may include a configuration for an insulin delivery device and the output may be provided to the insulin delivery device. The insulin delivery device may adjust an insulin output based on the machine learning output. According to an implementation, the external device may be configured in addition to configuring the light integrated device such that both the external device and the light integrated device are configured based on the machine learning output.

FIG. 7B depicts a system environment 720 for targeted light therapy in accordance with the subject matter disclosed herein. A light integrated device 722 may include one or more sensors 724, processor 726, memory 728, and dual LEDs 730. In some implementations, processors 726 may include one or more microprocessors, microchips, or application-specific integrated circuits. Memory 728 may include one or more types of random-access memory (RAM), read-only memory (ROM), and cache memory employed during execution of program instructions and may further include storage including one or more databases, cloud components, servers, or the like. The storage may include a computer-readable, non-volatile hardware storage device that stores information and program instructions. Processor 726 may use data buses to communicate with memory 728, sensors 724, and/or dual LEDs 730. Processor 726 and/or another component (e.g., a transmitter and/or receiver) associated with light integrated device 722 may be configured to transmit and/or receive data (e.g., sensed data, one or more configurations, etc.).

Analytics module 750 may be housed at light integrated device 722 or may be an external component, as shown in FIG. 7B. Analytics module 750 may include a processor 752 and a machine learning module 754. Machine learning module 754 may be a set of instructions, code, or the like that are implemented (e.g., compiled) using processor 752. An external analytics module 750 may communicate with light integrated device 722 via wired or wireless connection. The wireless connection may be via a network 740 such that analytics module 750 may be a remote or cloud component.

External sensors 760 may be an independent sensor or an external device including one or more sensors, where external sensors 760 or a respective external device is configured to communicate with light integrated device 722 and/or analytics module 750 via wired or wireless connection. External sensors 760 may include, but are not limited to, wearable sensors 762, fitness device sensors 764, cameras 766, intra-body sensors 768, or the like.

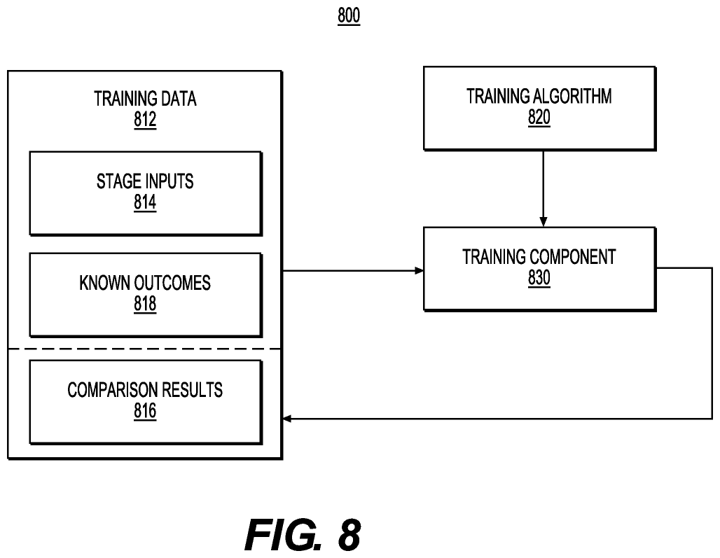

One or more implementations disclosed herein include a machine learning model. For example, as disclosed herein, a machine learning model may output operational parameters or settings to operate the light integrated device based on, for example sensor data regarding user health. A machine learning model disclosed herein may be trained using the data flow 800 of FIG. 8. As shown in FIG. 8, training data 812 may include one or more of stage inputs 814 and known outcomes 818 related to a machine learning model to be trained. The stage inputs 814 may be from any applicable source disclosed herein, including sensor data. The known outcomes 818 may be included for machine learning models generated based on supervised or semi-supervised training. An unsupervised machine learning model may not be trained using known outcomes 818. Known outcomes 818 may include known or desired outputs for future inputs similar to or in the same category as stage inputs 814 that do not have corresponding known outputs.

The training data 812 and a training algorithm 820 may be provided to a training component 830 that may apply the training data 812 to the training algorithm 820 to generate a machine learning model. According to an implementation, the training component 830 may be provided comparison results 816 that compare a previous output of the corresponding machine learning model to apply the previous result to re-train the machine learning model. The comparison results 816 may be used by the training component 830 to update the corresponding machine learning model. The training algorithm 820 may utilize machine learning networks and/or models including, but not limited to a deep learning network such as Deep Neural Networks (DNN), Convolutional Neural Networks (CNN), Fully Convolutional Networks (FCN) and Recurrent Neural Networks (RCN), probabilistic models such as Bayesian Networks and Graphical Models, and/or discriminative models such as Decision Forests and maximum margin methods, or the like.

In general, any process or operation discussed in this disclosure that is understood to be computer-implementable, such as communicating with an application via a GUI, adjusting light integrated device parameters or settings, etc., may be performed by one or more processors of a computer system. A process or process step performed by one or more processors may also be referred to as an operation. The one or more processors may be configured to perform such processes by having access to instructions (e.g., software or computer-readable code) that, when executed by the one or more processors, cause the one or more processors to perform the processes. The instructions may be stored in a memory of the computer system. A processor may be a central processing unit (CPU), a graphics processing unit (GPU), or any suitable types of processing unit.

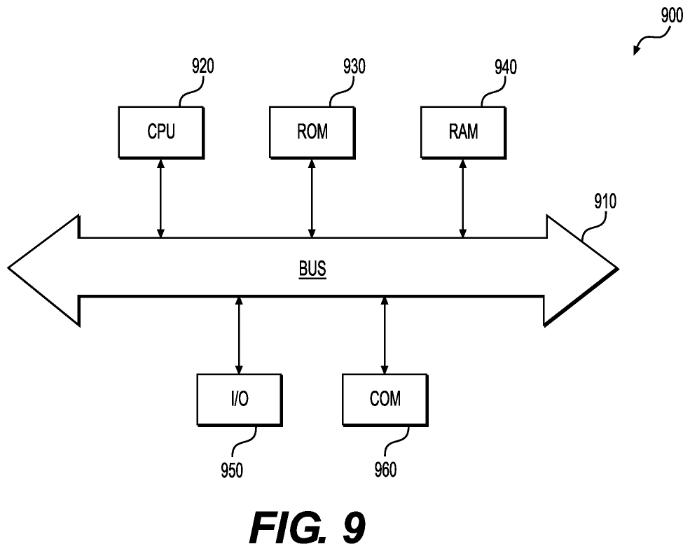

FIG. 9 depicts an example system 900 that may execute techniques presented herein. FIG. 9 is a simplified functional block diagram of a computer that may be configured to execute techniques described herein, according to exemplary embodiments of the present disclosure. Specifically, the computer (or “platform” as it may not be a single physical computer infrastructure) may include a data communication interface 960 for packet data communication. The platform may also include a central processing unit (“CPU”) 920, in the form of one or more processors, for executing program instructions. The platform may include an internal communication bus 910, and the platform may also include a program storage and/or a data storage for various data files to be processed and/or communicated by the platform such as ROM 930 and RAM 940, although the system 900 may receive programming and data via network communications. The system 900 also may include input and output ports 950 to connect with input and output devices such as keyboards, mice, touchscreens, monitors, displays, etc. Of course, the various system functions may be implemented in a distributed fashion on a number of similar platforms, to distribute the processing load. Alternatively, the systems may be implemented by appropriate programming of one computer hardware platform.

The general discussion of this disclosure provides a brief, general description of a suitable computing environment in which the present disclosure may be implemented. In one embodiment, any of the disclosed systems, methods, and/or graphical user interfaces may be executed by or implemented by a computing system consistent with or similar to that depicted and/or explained in this disclosure. Although not required, aspects of the present disclosure are described in the context of computer-executable instructions, such as routines executed by a data processing device, e.g., a server computer, wireless device, and/or personal computer. Those skilled in the relevant art will appreciate that aspects of the present disclosure can be practiced with other communications, data processing, or computer system configurations, including: Internet appliances, hand-held devices (including personal digital assistants (“PDAs”)), wearable computers, all manner of cellular or mobile phones (including Voice over IP (“VoIP”) phones), dumb terminals, media players, gaming devices, virtual reality devices, multi-processor systems, microprocessor-based or programmable consumer electronics, set-top boxes, network PCs, mini-computers, mainframe computers, and the like. Indeed, the terms “computer,” “server,” and the like, are generally used interchangeably herein, and refer to any of the above devices and systems, as well as any data processor.

Aspects of the present disclosure may be embodied in a special purpose computer and/or data processor that is specifically programmed, configured, and/or constructed to perform one or more of the computer-executable instructions explained in detail herein. While aspects of the present disclosure, such as certain functions, are described as being performed exclusively on a single device, the present disclosure may also be practiced in distributed environments where functions or modules are shared among disparate processing devices, which are linked through a communications network, such as a Local Area Network (“LAN”), Wide Area Network (“WAN”), and/or the Internet. Similarly, techniques presented herein as involving multiple devices may be implemented in a single device. In a distributed computing environment, program modules may be located in both local and/or remote memory storage devices.

Aspects of the present disclosure may be stored and/or distributed on non-transitory computer-readable media, including magnetically or optically readable computer discs, hard-wired or preprogrammed chips (e.g., EEPROM semiconductor chips), nanotechnology memory, biological memory, or other data storage media. Alternatively, computer implemented instructions, data structures, screen displays, and other data under aspects of the present disclosure may be distributed over the Internet and/or over other networks (including wireless networks), on a propagated signal on a propagation medium (e.g., an electromagnetic wave(s), a sound wave, etc.) over a period of time, and/or they may be provided on any analog or digital network (packet switched, circuit switched, or other scheme).

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method for configuring a light integrated device, the method comprising:
receiving sensed data from a first sensor;
providing the sensed data from the first sensor to a machine learning model;
receiving a machine learning output from the machine learning model based on the sensed data from the first sensor, the machine learning output comprising a light integrated device configuration, wherein the light integrated device configuration comprises a dual light emitting diodes (LED) setting; and
configuring the light integrated device based on the light integrated device configuration.

2. The method of claim 1, wherein the light integrated device comprises a dual LED and the dual LED setting includes activation of at least two different wavelengths from the dual LED.

3. The method of claim 2, wherein the activation of the at least two different wavelengths comprises one or more of an activation of a first wavelength and a second wavelength, an activation of the first wavelength at a first intensity and of the second wavelength at a second intensity, or an activation of the first wavelength for a first duration and of the second wavelength for a second duration.

4. The method of claim 2, wherein the at least two wavelengths are selected from a range of approximately 600 nm-1000 nm.

5. The method of claim 1, wherein the sensed data is one or more of biometric data, exhaled breath condensate (EBC) data, pH levels, saliva data, chemical data, shape data, object data, electrical mitochondria data, protein data, glucose data, lactate data, urea data, serum data, blood data, light data, biochemical data, electrochemical data, volatile organic compounds (VOCs) biomarker data, laser data, force data, or movement data.

6. The method of claim 1, wherein the machine learning model is trained based on cohort data, wherein the cohort data is based on a plurality of cohort users.

7. The method of claim 1, wherein the light integrated device configuration comprises further comprises one or more of intensities of light, rates, durations, or frequencies for configuring the light integrated device.

8. The method of claim 1, further comprising:
receiving sensed data from a second sensor;
providing the sensed data from the second sensor to the machine learning model; and
receiving an updated machine learning output from the machine learning model based on the sensed data from the second sensor.

9. The method of claim 1, further comprising:
receiving updated sensed data from the first sensor after configuring the light integrated device based on the light integrated device configuration;
providing the updated sensed data from the first sensor to the machine learning model;
receiving an updated machine learning output from the machine learning model based on the updated sensed data from the first sensor, the updated machine learning output comprising an updated light integrated device configuration; and
configuring the light integrated device based on the updated light integrated device configuration.

10. The method of claim 1, wherein the machine learning output further comprises an external component configuration and further comprising outputting the external component configuration to an external component.

11. A light integrated device comprising:
a housing;
one or more sensors associated with the housing;
one or more dual light emitting diodes (LEDs) associated with the housing, wherein each dual LED is configured to output a first light having a first wavelength and a second light having a second wavelength; and
a processor configured to cause the one or more dual LEDs to operate based on a light integrated device configuration.

12. The light integrated device of claim 11, wherein the light integrate device configuration is output by a machine learning model.

13. The light integrated device of claim 12, wherein:
the one or more sensors are configured to sense sensed data;
the processor is configured to apply the sensed data as an input to the machine learning model; and
the machine learning model is configured to output the light integrate device configuration based on the sensed data.

14. The light integrate device of claim 11, wherein the one or more dual LEDs fluctuate at a frequency of less than approximately 3 Hz.

15. The light integrate device of claim 11, wherein the housing is one of a patch, a shower head, an exercise equipment, a bulb, a transcranial device, a head gear, a mask, an eyewear, a wearable device, or an intra-body device.

16. A system for providing light therapy to a user, the system comprising:
one or more sensors configured to sense sensed data;
a light integrated device comprising one or more dual light emitting diodes (LEDs), wherein each dual LED is configured to output red light and near red light;
at least one memory storing instructions; and
at least one processor executing the instructions to perform a process, the processor configured to:
receive the sensed data sensed by the one or more sensors;
receive a light integrated device configuration based on the sensed data, the light integrated device configuration comprising one or more of wavelengths of light, intensities of light, rates, durations, or frequencies for configuring the light integrated device; and
configure the light integrated device based on the light integrated device configuration.

17. The system of claim 16, wherein the light integrated device configuration is generated by a machine learning model based on the sensed data.

18. The system of claim 16, wherein the processor is further configured to:
apply the sensed data as an input to a machine learning model; and
receive a machine learning output from the machine learning model based on the sensed data, the machine learning output comprising the light integrated device configuration.

19. The system of claim 16, wherein the processor is further configured to:
transmit the sensed data over a network; and
receive the light integrated device configuration from the network.

20. The system of claim 16, further comprising an analytics module comprising a machine learning model configured to generate the light integrated device configuration based on the sensed data.
